(19) United States
(12) Patent Application Publication
(10) Pub. No.: US 2020/0330379 A1
(43) Pub. Date: Oct. 22, 2020

(54) NON-ORAL CANNABINOID FORMULATION AND METHOD OF TREATMENT

(71) Applicant: Navinta NV, Inc., Las Vegas, NV (US)

(72) Inventors: Dhirender Singh, Ewing, NJ (US); Ramakrishna Borra, Bensalem, PA (US); Sandipkumar Arvindbhai Patel, Robbinsville, NJ (US)

(21) Appl. No.: 16/850,536

(22) Filed: Apr. 16, 2020

Related U.S. Application Data
(60) Provisional application No. 62/835,120, filed on Apr. 17, 2019.

(51) Int. Cl. A61K 9/06; A61K 31/05; A61K 31/352; A61K 47/38

(52) U.S. Cl. CPC A61K 9/06; A61K 31/05; A61K 9/0031; A61K 47/38; A61K 31/352

(57) ABSTRACT

A pharmaceutically acceptable formulation of cannabinoid for non-oral delivery. The formulation including at least one cannabinoid at 0.05% to 50% by weight of the total formulation. The formulation being delivered to a patient to alleviate symptoms including, but not limited to, pain, nausea, vomiting, and the like.

### CROSS-REFERENCE TO RELATED APPLICATION(S)

The instant application claims priority to U.S. Provisional Patent Application No. 62/835,120, entitled “Non-Oral Cannabinoid Formulation and Method of Treatment,” filed on Apr. 17, 2019, the entirety of which is incorporated by reference herein.

### FIELD OF THE INVENTION

This invention relates to formulations of cannabinoids for medical use. In particular, formulations according to embodiments herein are delivered to a non-oral cavity, which facilitates effective absorption of active ingredients through a mucosal membrane, while circumventing the extensive first pass hepatic metabolism, thereby reducing dose burden.

### BACKGROUND

The cannabis plant has many naturally occurring diverse chemical compounds called cannabinoids, that in recent years, have seen a resurgence in interest in the therapeutic potential. In general, cannabinoid is a type of chemical in hemp or cannabis plant that causes drug-like effects all through the body, including the central nervous system and the immune system.

To date, more than 140 cannabinoids have been identified and isolated from the cannabis plant. However, two major neuroactive components, i.e., the non-psychoactive cannabidiol (CBD), and psychoactive Δ9-tetrahydro-cannabinol (Δ9-THC), have been investigated and have potential of great medicinal use. The cannabinoids act mainly by altering neurotransmitter release, upon binding to receptor sites throughout the brain (CB1 receptors) and body (CB2 receptors).

The IUPAC nomenclature of CBD is 2-[(1R,6R)-6-Isopropenyl-3-methylcyclohex-2-en-1-yl]-5-pentylbenzene-1,3-diol. The IUPAC nomenclature for THC is (6aR,10aR)-6,6,9-trimethyl-3-pentyl-6a,7,8,10a-tetrahydro-6H-benzo[c]-chromen-1-ol. Cannabichromene (CBC) has the IUPAC nomenclature of 2-methyl-2-(4-methylpent-3-enyl)-7pentyl-5-chromenol. Cannabigerol (CBG) has the IUPAC nomenclature of 2-[(2E)-3,7-dimethylocta-2,6-dienyl]-5-pentyl-benzene-1,3-diol. These are among the most prominent cannabinoids in the family of chemical compounds extracted from the cannabis plant, however, CBD is the major constituent of the cannabis plant, comprising more than 40% of the extracts. General chemical structures and numbering system for CBD and Δ9-THC are illustrated in FIG. 1.

Currently, synthetic cannabinoids are being investigated extensively for medicinal purposes.

To date, CBD and THC have been investigated for potential treatment in a vast majority of disease indications including, seizure, pain, inflammation, arthritis, fatigue, muscle weakness, tremors, nausea, vomiting, and lack of appetite sarcopenia, cachexia, AIDS wasting. Studies show the efficacy of CBD in the treatment of central nervous system (CNS) disease acting as anticonvulsant, antinociceptive, antipsychotic, neuroprotective antioxidant activity, muscle relaxant, sedation, catalepsy; and used in cardiovascular disease, e.g., bradycardia, hypotension. THC has been reported as efficacious for CNS aliments, nociceptive, catalepsy, psychotropic, emetic, antioxidants and neuroprotectants, sedation and cardiovascular disease e.g., tachycardia, hypertension.

However, chronic use of cannabinoids leads to severe cannabis-abuse, specifically upon raw plant or plant smoke consumption. Collectively, this abuse is referred to as “cannabis use disorder”. Discontinuance of cannabis consumption can result in withdrawal symptoms. However, some cannabinoids, such as CBD, may be useful in treating cannabis use disorder. Various methods to deliver CBD or other cannabinoids are available, aiming at avoiding inhalation of burning materials.

Recently, The U.S. Food and Drug Administration approved the oral solution of CBD (marketed as EPIDIOLEX®), at 100 mg/mL strength, for the clinical indication of seizures associated with Lennox-Gastaut syndrome or Dravet syndrome in patients 2 years of age and older. Unfortunately, as described in the EPIDIOLEX® PIL, the higher doses of EPIDIOLEX® increase the risk of transaminase elevations that leads to severe hepatocellular Injury. Hence, it is important to have lower dose titration in patients with moderate or severe hepatic impairment.

CBD is extensively metabolized by the liver, approximately 60% of clinically prescribed drug is metabolized via CYP3A4 that are outlined the “Hepatocellular Injury” in the PIL of EPIDIOLEX®. Moreover, since, CBD and THC are subjected to a significant first-pass effect, the bioavailability of CBD and THC is reduced significantly when administered orally. Oral bioavailability of CBD was reported to be 13-19%, mainly due to first-pass metabolism. Likewise, predominantly hindered due to first-pass metabolism, the oral bioavailability of THC is limited to a range in between 10% and 20%. Consequently, a very high dose is required to attain therapeutic benefits.

Due to low bioavailability of oral CBD and THC formulations, alternative routes of administration, including sublingual or mucosal, inhalation, and rectal, have been investigated to circumvent the hepatic first-pass effect. Circumventing first-pass metabolism significantly reduces high dose burden while possessing similar pharmacological effectiveness with a comparatively lower dose.

To this context, an oromucosal spray consisting of delta-9-THC and CBD in a 1:1 ratio is clinically used in multiple sclerosis (MS) to improve symptoms related to muscle stiffness, called “spasticity”. However, the route allows only a limited amount of drug to be administered, thus restricting the use in indication where low dose is required. Moreover, the sublingual dosage stimulates saliva production which make it difficult for patient to hold the formulation into mouth. Once swallowed the drug is subjected to poor absorption and first-pass metabolism.

Like oromucosal delivery, nasal administration can improve the bioavailability, however, it may cause pain or reflex sneezing. In extreme cases it may cause damage or irritation to the nasal mucosa. Moreover, this route is also restricted to administration of small dose.

Recently, rectal administration has seen a surge in delivery of therapeutics, that suffer from severe first-pass metabolism effect and negate the concomitant requirement of a higher dose. Moreover, rectal administration is beneficial where other routes are ineffective, invasive, impractical or too difficult to administer to be effective.

A suppository is a solid fixed-dose medical preparation, formulated in a cylindrical or conical shape, and designed to be inserted into the rectal cavity. A suppository is designed to melt inside the rectum by body heat, and concomitantly dissolve in a bodily fluid. However, a major drawback of suppository dosage forms is that it does not permit dose adjustment, which is a key requirement for many indications. Moreover, suppositories are slow to melt, and therefore, the time it takes for the therapeutic agent to be absorbed may be unreasonably long for an indication that requires immediate effect, for instance, seizures that are in progress.

Accordingly, what is needed in the art is a formulation that can be delivered by a mechanism that provides a much faster absorption of the cannabinoid, compared with oral or suppository dosage, in its capacity of eliminating the additional step of disintegration or melting of the active moiety from solid or suppository dosage into solution. Moreover, a formulation is needed that can be administered non-orally, e.g., rectally, using an appropriate applicator, can be a potential dosage form circumventing the dose titration limitation of a suppository dosage form. What is further needed in the art is a formulation having a dosage to achieve the therapeutic effectiveness with a much smaller dose as compared to formulations currently known in the art, while minimizing the dose-dependent adverse effects.

### SUMMARY OF THE INVENTION

A liquid, viscous liquid, or gel formulation of one or more cannabinoids is disclosed herein. In one embodiment a liquid, viscous liquid or gel formulation of CBD or/and THC is disclosed, which is formulated with biocompatible and compendial approved excipients. An advantage of such formulation is much simpler formulation that can be administered to a cavity other than oral or transdermal, preferably rectal or vaginal. Such novel formulation is suitable for indications where dose adjustment is required, for example, epileptic seizures. Moreover, an aqueous based formulation immediately dissolves in bodily fluid, and further improves the onset of action and bioavailability. Non-oral administration of cannabinoids circumvents the extensive first-pass metabolism, and further reduce the high dose-burden and dose-associated adverse effects, for instance, hepatocellular. To this end, proposed formulations would be an ideal dosage to achieve the therapeutic effectiveness with much smaller dose, while minimizing the dose-dependent adverse effects. Moreover, the disclosed formulations are effective where oral administration is ineffective, difficult or impractical, for example, nausea, vomiting and patient with active seizure

In one embodiment, the invention is directed to a pharmaceutically acceptable gel formulation of cannabinoid for non-oral delivery comprising: (a) at least one cannabinoid at 0.05% to 50% by weight of the total formulation; (b) optionally a pH adjusting agent in a concentration effective to maintain the pH of the formulation at about 5-8; (c) a thickener in a concentration effective to impart a viscosity to the formulation in a range of 1,000 to 8,000 Centipoise (cP) so that it is suitable for administration by applicator or syringe like injector to a human patient; and (d) a solvent at 10% to 95% by weight of the total formulation.

In one embodiment, the at least one cannabinoid is selected from the group consisting of CBD, CBDA, CBDV, THC, THCA, THCV, CBG, CBN, and combinations thereof.

In one embodiment the at least one cannabinoid is CBD and wherein CBD comprises 0.1% to 20% by weight of the total formulation. In another embodiment, the at least one cannabinoid is THC and wherein THC comprises 0.05% to 2% by weight of the total formulation. In another embodiment, the at least one cannabinoid comprises a mixture of CBD and THC, wherein CBD comprises 0.1% to 20% by weight of the total formulation, and THC comprises 0.05% to 2% by weight of the total formulation.

In one embodiment, the concentration of the thickener is 0.05 to 10% by weight of the total formulation. The thickener is selected from the group consisting of a cellulose ether; a polyacrylic acid polymer (carbomer); natural gum, and combinations thereof. The cellulose ether is selected from the group consisting of hydroxypropyl methylcellulose, methylcellulose, hydroxypropyl cellulose, carboxyl methylcellulose, and combinations thereof. In a particular embodiment, the thickener is hydroxypropyl methylcellulose.

In one embodiment, the solvent is water or a mixture of water and at least one organic solvent.

In one embodiment, the formulation further comprises a pharmaceutically acceptable excipient selected from group consisting of preservatives, buffers, inclusion complexing agents, surfactants and combinations thereof.

In one embodiment the at least one cannabinoid is suspended in the gel composition. A size of the suspended cannabinoid particle is between 10 nm (Nanometer) to 50 μm (Micrometer).

The formulation is suitable for administration to a patient via a rectal route of administration. The formulation is packaged in a suitable rectal applicator. The formulation is suitable for administration in a non-oral cavity of a patient. The formulation is suitable for transdermal application.

In one embodiment, the invention is directed to a pharmaceutically acceptable liquid or viscous liquid formulation of cannabinoid for non-oral delivery comprising:
a. at least one cannabinoid at 0.05% to 50% by weight of the total formulation;
b. optionally an antioxidant;
c. optionally a pH adjusting agent; and
d. optionally an alcohol-based co-solvent

wherein the formulation is suitable for rectal or vaginal administration.

In one embodiment, the liquid or viscous liquid formulation further comprises a pharmaceutical excipient; or a hydrogenated oil at 10% to 95% by weight of the total formulation.

In one embodiment of the liquid or viscous liquid formulation, the at least one cannabinoid is selected from the group consisting of CBD, CBDA, CBDV, THC, THCA, THCV, CBG, CBN and combinations thereof. In a particular embodiment of the liquid or viscous liquid formulation, the at least one cannabinoid is: CBD; THC; or a combination of CBD and THC.

One embodiment is directed to a kit for administering a formulation of cannabinoid for non-oral delivery, the kit comprising: a syringe assembly pre-filled with a dose of a formulation according to embodiments herein; and instructions of use, wherein the syringe assembly and instructions are placed in packaging.

In another embodiment, the invention is directed to a method for administering a formulation of cannabinoid to alleviate symptoms exhibited by a patient, the method comprising: providing a formulation of cannabinoid, the formulation comprising: at least one cannabinoid at 0.05% to 50% by weight of the total formulation; and optionally a pH adjusting agent in a concentration effective to a maintain the pH of the formulation at about 5-8; and delivering the formulation to a non-oral cavity of the patient, wherein after said delivery of the gel formulation, the symptoms of the patient are alleviated.

These and other embodiments are disclosed in more details herein.

### BRIEF DESCRIPTION OF THE FIGURES

FIG. 1 illustrates known general chemical structures and numbering system for cannabidiol (CBD) and Delta-9-tetrahydrocannabinol (THC).

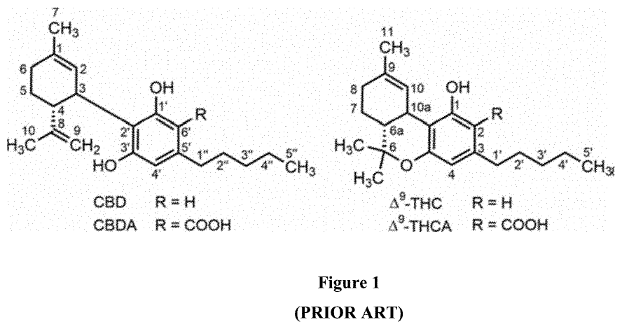

FIG. 2 illustrates an applicator that is useful in dispensing (administering) a formulation as disclosed herein.

FIG. 3 illustrates a kit according to embodiments described herein.

### DETAILED DESCRIPTION OF THE INVENTION

The present invention relates to a pharmaceutically acceptable formulation of cannabinoids. The term “pharmaceutically acceptable” means, e.g., that it is appropriate to administer to a human patient, includes minimal or no contaminants, and/or is in a form that is acceptable to deliver to a human patient.

In one embodiment, the formulation is a liquid solution. In one embodiment, the formulation is viscous, aqueous gel formulation. In one embodiment, the formulation is a viscous, non-aqueous gel formulation. In one embodiment, the formulation is an oleo gel formation. In one embodiment, the formulation is a viscous, emulsion formulation. In one embodiment, the formulation is a viscous, suspension formulation. In another embodiment, the present invention relates to a viscous liquid formulation.

In one embodiment, the formulation is administered into a non-oral cavity or transdermally. The formulation is useful to significantly lower the dose burden of cannabinoid by circumventing first-pass hepatic metabolism that is associated with oral dosage administration. Moreover, the gel or liquid formulation is useful where another delivery form or delivery route, e.g., oral delivery, is ineffective, invasive, or impractical. The gel and liquid formulations disclosed herein are useful where dose adjustment and titration are required. The inventors have surprisingly discovered that the gel and liquid formulations according to the embodiments herein can effectively and efficiently deliver dosages of cannibinoids to a patient in need of treatment. It has surprisingly been found that the formulations disclosed herein have the benefit of rectal suppositories (i.e., the bioavailability) without the problems associated therewith. Similarly, the formulations according to the present invention are beneficial over other known modes of delivery, e.g., oral tablets, smoking, suppositories, since the current formulations can be delivered in a safe and efficient manner.

### A. FORMULATION

The present invention is a formulation of one or more cannabinoids suitable for administration to a patient through a non-oral cavity, e.g., delivery (administration) vaginally, rectally, otic (ear), or nasally. In certain embodiments, the formulation is a gel, an emulgel, an oleo gel, a microemulsion, a solution (i.e., a liquid), a viscous liquid, or a self-emulsifying solution suitable to form an emulsion after contact with water or aqueous fluid. In one embodiment, the formulation is a gel. In another embodiment the formulation is an aqueous gel. In another embodiment the formulation is a non-aqueous gel. In another embodiment the formulation is a liquid. In another embodiment the formulation is a viscous liquid. Examples of liquid or viscous liquid formulations include those formulations that are identified as a microemulsion, a self-emulsifying solution, an emulgel, a liquid, and a viscous liquid.

A particular embodiment is a gel formulation of CBD, THC, or a combination thereof in an aqueous or non-aqueous viscous liquid that is suitable for vaginal and/or rectal administration. The gel formulation may further comprise alcohol (including modified alcohol such as but not limited to polyethylene glycol, propylene glycol, glycerol), water, a surfactant, a penetration enhancer, an antioxidant, a pH adjusting agent, a viscosity enhancing polymer, and any combination of the foregoing.

An emulgel (an emulsion with thickening agent in continuous phase) disclosed in this invention is a viscous oil in water or water in oil emulsion where the drug is solubilized or dispersed in either or both phases. An oleo gel disclosed in this invention is viscous oil-based formulation. Oleo gel comprises a water immiscible base as a vehicle.

A microemulsion disclosed in this invention is multiple phase composition containing water miscible phase and oil miscible phase. Microemulsions can be oil in water, water in oil, oil in water in oil or water in oil in water. Microemulsions further comprise oil, alcohol (including modified alcohol such as but not limited to polyethylene glycol, propylene glycol, glycerol), water, surfactant, penetration enhancer, antioxidant, pH adjustment agent, and any combinations of the foregoing.

A solution (liquid) disclosed in this invention is an aqueous or non-aqueous solution suitable for rectal and/or vaginal administration. Solutions may comprise any of oil, alcohol (including modified alcohol such as but not limited to polyethylene glycol, propylene glycol, glycerol), water, surfactant, penetration enhancer, antioxidant, pH adjustment agent or any combinations of the foregoing.

### B. CANNABINOIDS

The formulations disclosed herein include at least one cannabinoid. To date, more than 140 cannabinoids have been identified and isolated from the cannabis plant. However, two major neuroactive components i.e. cannabidiol (CBD), and Δ9-tetrahydro-cannabinol (Δ9-THC), have been investigated extensively in the field of medicine. A plethora of studies have been published investigating potential of these two cannabinoids in the treatment of vast array of medical conditions, primarily, but not limited to, amyotrophic lateral sclerosis (ALS), pain, inflammation, arthritis, nausea, vomiting, and lack of appetite, convulsions, nociceptive pain, psychosis, oxidative stress, muscle tension, sedation, anxiety catalepsy and cardiovascular disease.

The inventors surprisingly found that a formulation containing a cannabinoid or a combination of cannabinoids can be prepared that is suitable for non-oral route of administration. The cannabinoid is selected from: CBC (Cannabichromene), CBD (Cannabidiol), CBDA (Cannabidiolic acid), CBDV (Cannabidivarin), CBG (Cannabigerol), CBN (Cannabinol), THC (Tetrahydrocannabinol), THCA (Tetrahydrocannabinolic acid), THCV (Tetrahydrocannabidivarin), and combinations thereof.

One embodiment provides a formulation that includes at least one cannabinoid selected from CBD, CBDA, CBDV, THC, THCA, THCV, CBG and CBN.

In a preferred embodiment, the formulation comprises at least one cannabinoid at 0.05% to 50% wt. of the total formulation. In another embodiment, the formulation comprises at least one cannabinoid at 0.05% to 45% wt.; 0.05% to 40% wt.; 0.10 to 40% wt.; 0.30 to 35% wt.; 0.5 to 30% wt.; 0.5 to 25% wt.; 0.5 to 20% wt.; 0.5 to 15% wt.; 1.0 to 40% wt.; 1.0 to 30% wt.; 1.0 to 20% wt.; 1.0 to 10% wt. based on the total weight of the formulation.

In one embodiment, CBD is the only cannabinoid in the formulation, wherein the concentration of CBD is about 0.1-50% wt. of the formulation. In one embodiment, the concentration of CBD is about 0.1-20% wt. of the formulation, preferably about 0.5-to about 20% wt. In one embodiment, THC is the only cannabinoid in the formulation, wherein the concentration of THC is about 0.1-50% wt. In one embodiment, the concentration of THC is about 0.05% to 2% wt. of the total formulation.

In a particular embodiment, both CBD and THC are used in the formulation, wherein the concentration of CBD is about 0.1-20 wt. % of the formulation, and the concentration of THC is about 0.05% to 2% wt. of the total formulation.

### C. THICKENER

The gel or viscous liquid formulation of the present invention typically contains a thickener effective for rendering the consistency of the formulation appropriate for administration to a non-oral cavity by suitable applicator. To be effectively administered as a rectal injectable, the present gel formulation has a viscosity such that it can be administered easily by injection, however once administered, should not leak out from the cavity. If the viscosity of the formulation is too low, with body movement the formulation may leak out of the cavity after administration. Such a leakage would compromise dose and desired therapeutic effect. A high viscosity of the formulation would render it difficult for administration into the non-oral cavity of a patient by injection using the applicator.

In one embodiment the gel or viscous liquid formulation has an apparent viscosity of about 1,000 to about 8,000 centipoise (cP), and more preferably, about 1500 to about 5,000 cp.

The desired viscosity of the formulation may be achieved by including a physiologically-acceptable thickener (also known as a thickening agent) in the formulation. Representative examples of thickeners include but are not limited to cellulose ethers, such as carboxy-methylcellulose and methylcellulose, hydroxypropylmethylcellulose (HPMC), e.g., Methocel E50 LVP, K100LVP, E4MP, hydroxypropyl cellulose, e.g., KLUCEL LF, GF, MF; carboxy vinyl polymers, e.g., Carbomer or Carbopol; biogums e.g., xanthan gum; and combinations thereof.

Generally, an effective amount or concentration of a suitable thickener is used to provide a viscosity within the desired range. In one embodiment, the concentration of thickener is about 0.05 to 10% wt. of the total formulation. In another embodiment, the concentration of thickener is about 0.05 to 7% wt.; 0.1 to 7% wt.; 0.5 to 7% wt.; 1 to 7% wt.; 2 to 7% wt.; 0.05 to 2% wt.; 0.1 to 2% wt.; 0.2 to 2% wt.; 0.2 to 1% wt. of the total formulation. In a particular embodiment, cellulose ether is a thickener and is present in a concentration of about 1-10 wt. % of the formulation, preferably about 2 to 7% wt. In another embodiment, carboxyl vinyl polymer is the thickener at a concentration of about 0.05 to 2% wt., preferably about 0.2 to 1% wt. of the total formulation.

### D. PH ADJUSTING AGENT

The natural pH in the non-oral cavity is about 7.0 which is considered a neutral pH. The pH of the formulation can be maintained at a neutral pH, or any other desired pH, using a suitable pH adjusting agent. A pharmaceutically acceptable pH adjusting agent (i.e., a buffer system) can include an organic acid and the corresponding potassium or sodium salt, e.g., acetic acid-sodium acetate, citric acid-sodium citrate, and benzoic acid-sodium benzoate, preferably benzoic acid-sodium benzoate.

The concentration of the pH adjusting agent is about 0.01 to 10% wt. of the formulation. In one embodiment the concentration of the pH adjusting agent is about 0.01 to 9% wt. of the formulation. In another embodiment, the concentration of the pH adjusting agent is about 0.05 to 9% wt.; 0.05 to 5% wt.; 0.1 to 9% wt.; 0.1 to 8% wt.; 0.2 to 8% wt.; 0.2 to 5% wt.

In one embodiment, the pH of the formulation is from about 5 to about 8. Preferably the pH of the formulation is from about 5.5 to about 7.5. In a more preferred embodiment, the pH of the formulation is from about 6 to about 7. In another preferred embodiment, the pH of the formulation is from about 5.5 to about 6.5.

In a preferred embodiment of the present formulation, the pH adjusting agent is benzoic acid. The concentration of benzoic acid is about 0.01 to 5 wt. % of the final formulation. In another embodiment, the pH adjusting agent is sodium benzoate at a concentration of about 1 to about 5 wt. % of the formulation to maintain the pH at about 5 to about 8.

### E. SOLVENT

In some embodiments of the formulation, i.e., aqueous gel formulations, liquid (solution) formulations, viscous liquid formulations, and the like, a solvent is present. In one embodiment, the solvent is water. In another embodiment, the solvent includes water and an additional solvent. In yet a further embodiment, the solvent includes water and more than one additional solvent, such as, e.g., an organic solvent. In embodiments of the formulations that take the form of oleo gels, self-emulsifying solutions and the like, there are no solvents present.

In one embodiment, the total concentration of solvents is between 5% to 95% by weight of the total formulation. In another embodiment, the total concentration of solvent(s) is between 5% to 90% by weight; between 5% to 80% by weight; between 10% to 70% by weight; between 10% to 60% by weight; between 15% to 50% by weight; between 20% to about 40% by weight, based on the total formulation weight.

### (i) Water

In some embodiments of the formulation, water acts as vehicle for the ingredients (i.e., components) present in the formulation. Water from any available source can be used. Preferably, the water is distilled or deionized water. In one embodiment, water can constitute about 5 to 95% wt. of the gel formulation, most preferably, about 10 to 50% wt.; 10 to 25% wt.; 15% wt. of the gel formulation. Having added suitable thickening agents selected in above mentioned concentration range, the present invention yields a flowable viscous gel consistency.

### (ii). Organic Solvent

In one embodiment, the formulation may include water and an organic solvent. Depending on the particular cannabinoid, different organic solvents may be used. In one embodiment, nontoxic polyols or alkanols such as propylene glycol and/or ethyl alcohol may be used as the organic solvent.

An organic solvent may function as a penetration enhancer, which may facilitate the absorption of the cannabinoid by the mucosal membrane. A penetration enhancer may be included in some embodiments of the formulation. Ethanol and polyols such as glycerol and propylene glycol can act as a penetration enhancer.

If used in the formulation, the organic solvent or mixture of organic solvents are used in amounts effective to solubilize the cannabinoids and to inhibit precipitation thereof in the gel formulation. In one embodiment, the formulation has a concentration of between 0 to about 80% wt. of organic solvent (to function as a solvent and/or a penetration enhancer). In another embodiment, the formulation has an organic solvent concentration of between 5 to 80% wt.; about 10 to 75% wt.; about 10 to 60% wt.; about 15 to 75% wt.; about 20 to 50% wt. In one embodiment, the formulation has a concentration of about 5 to 80% wt. of a polyol or polyol-alkanol mixture. In a preferred embodiment, propylene glycol is present at a concentration of about 20 to about 60% wt. of the formulation. In another embodiment, ethyl alcohol is present at a concentration of about 5 to about 50% wt. In some cases, the solvent used in the present gel formulation can consist entirely of one or more nontoxic organic solvents.

Generally, a relatively clear formulation is preferred that is free from and undissolved particulate of drug or excipient moiety. Therefore, the selection of the solvent, their concentrations, and the pH of the final gel formulation is preferably done in a manner consistent with maintaining the clear formulation, thereof free of any precipitant.

### F. SOLUBILIZER

To achieve maximum absorption and bioavailability, the cannabinoids may be present in solution (liquid) form. However, some of the cannabinoids may not be readily soluble in water. Cyclodextrins (CD) have been employed for decades as a potential tool to increase the solubility and improve the bioavailability of the hydrophobic drug moiety. Generally, CDs are a family of cyclic oligosaccharides consisting of glucopyranose subunits linked through α-1,4 glycosidic bonds. CD possess hydrophobic interior and hydrophilic exterior that creates an intramolecular cage-like structure where hydrophobic drug compounds form a complex within hydrophobic cavity of CD, without forming covalent linkage. Alpha-, beta-, and gamma-cyclodextrin comprises 6, 7 and 8 glucose subunits respectively, are generally recognized as safe by the U.S. FDA.

In certain embodiments, one or more cannabinoids are solubilized in final gel formulation using alpha-, beta-, or gamma-CD, or their modified derivative. Preferably, beta-CD or derivative e.g., Sulfobutylether-β-CD (trade name Captisol) is used. Generally, a CD is present in the gel formulation at a molecular ratio of CD: Cannabinoid; 1:0.5 to 1:5, more preferably at a ratio 1:1 with one or more cannabinoid.

### G. PHARMACEUTICALLY ACCEPTABLE EXCIPIENTS

Some embodiments of the formulations described herein include one or more pharmaceutically acceptable excipients. In one embodiment, a pharmaceutically acceptable excipients include one or more of preservatives, inclusion complexing agent, and surfactants.

A biocompatible preservative is optionally included in the formulation to extend the shelf-life of the gel formulation against bacterial attack. Various safe and efficacious preservatives such as methyl parabens, propyl parabens, thimerosal, benzalkonium chloride, chlorobutanol, and benzyl alcohol can be used. Benzyl alcohol, which has already been approved by the FDA for rectal use, is a preferred preservative.

The final concentration of the preservative in the formulation depends on the type of preservative selected. In one embodiment, the preservative is present in the formulation at a concentration about 0.01% wt. to about 10% wt. of the total formulation. In another embodiment, the preservative is present in the formulation at a concentration of about 0.01% wt. to about 5% wt. of the total formulation.

In one embodiment, the preservative is present in the gel formulation at a concentration of about 0.01% wt. to about 3% wt. of the final gel formulation. In a specific example, the preservative is benzyl alcohol in a concentration from about 0.05% wt. to about 2.5% wt.

In some embodiments, the inclusion complexing agents include beta cyclodextrin, hydroxypropyl beta cyclodextrin, sulfobutyl-ether-betacyclodextrin, or pharmaceutically acceptable salts thereof. In some embodiments, the concentration of the inclusion complexing agent is from 5% to about 95%. In preferred embodiments, the concentration of the inclusion complexing agent is between 10% to 70%.

Surfactants, which are generally an amphipathic substance consisting of hydrophobic and hydrophilic groups may be an important component in pharmaceutical formulations. Depending on their charge content, surfactant molecules may be cationic, anionic, zwitterionic (ampholytic) or non-ionic. Depending upon characteristics, such as surface charge and HLB value, a surface-active agent selected from a particular group provide the majority of functionality in formulation e.g.; to improving the solubility of hydrophobic compound; to improve drug penetration and absorption; to improve bactericidal activity of formulation against a wide range of gram-positive and some gram-negative organisms. Examples of surfactants that can be used in gel formulation but are not limited to; phospholipids and derivatives; non-ionic surfactant e.g., Sorbitan esters, polysorbates Poloxamers, etc.; cationic surfactant e.g., quaternary ammonium and pyridinium cationic surfactants; anionic-surfactant e.g. sodium alkyl sulfates.

It is contemplated that the formulation may include one or more surfactants. Depending on the surfactant(s) selected for use in the formulation, the concentration of surfactant is from about 0.05% to 80% wt. of the total formulation. In another embodiment, the concentration of surfactant is from 0.05% to 25% wt. of the total formulation. In another embodiment, the concentration of surfactant is from about 0.05% to 20% wt. of the total formulation. Other concentrations are contemplated and include, about 0.05% to 5% wt.; 0.05% to 4% wt; from 0.1 to 4% wt.; from 0.1 to 1% wt of the total formulation.

### H. PENETRATION ENHANCER

Formulations according to embodiments herein may include one or more penetration enhancers. Penetration enhancers help increase the permeability of cannabidiol through biological membranes and thus improve the bioavailability of the canabidiol. Some of the known penetration enhancers that are incorporated in the formulations include, but are not limited to alkyl saccharides, Salcaprozate Sodium, Salcaprozic acid, Sodium caprate, chitosan, ethanol, polyols (e.g., glycerol, propylene glycol), and combinations thereof.

It is contemplated that the formulation may include one or more penetration enhancers. Depending on the penetration enhancer selected for use in the formulation, the concentration of the penetration enhancer is about 0.05% to 10% wt. of the total formulation. In another embodiment, the concentration of surfactant is from 0.05% to 5% wt; from 0.1 to 5% wt.; from 0.1 to 1% wt of the total formulation.

### I. HYDROGENATED OIL

In some embodiments of the formulation, and in particular liquid formulations, a further component is a hydrogenated oil. In some embodiments of the formulation, hydrogenated coconut oil, hydrogenated palm kernel oil, hydrogenated castor oil, hydrogenated cottonseed oil, hydrogenated corn oil, hydrogenated palm oil, hydrogenated soyabean oil, hydrogenated vegetable oil, or modified vegetable oil such as but not limited to polyoxyl 40-hydrogenated castor oil and polyoxyl-35 hydrogenated castor oil. Hydrogenated oils act as surfactant and improve the absorption of drug.

In one embodiment, the hydrogenated oil is present in the liquid formulation at a concentration of from about 10% to about 95% wt of the formulation. In another embodiment, the hydrogenated oil is present in the liquid formulation at a concentration of from 10% to about 80% wt.; about 15% to about 70% wt.; about 15% to about 60% wt.; about 15% to about 50% wt.; about 15% to about 30% wt.

### J. USES OF FORMULATION

Formulations according to embodiments described herein are used in the treatment of human patients. Treatment includes decreasing or eliminating (“alleviating”) symptoms or signs of a disease or condition. The formulations are used for treatment of various diseases, symptoms, and conditions that present in human patients. Diseases, symptoms and conditions that are treated by the formulations disclosed herein include, but are not limited to: nausea or vomiting (including, but not limited to vomiting and nausea due to chemotherapy); pain (e.g., pain associated with Multiple Sclerosis (MS), Menstrual pain, muscle spasms, muscle injuries, skeletal injuries, and cancer, etc.); post-operative ileus; intestinal inflammation (including inflammation caused by parasites, infections, irritable bowel syndrome, or inflammatory bowel disease, Crohn's disease, ulcerative colitis, etc.); CNS disorders (including, but not limited to amyotrophic lateral sclerosis (ALS), Seizure, convulsant, nociceptive, psychotic, oxidative stress, sedation, and catalepsy); AIDS wasting; cachexia and sarcopenia; arthritis; fatigue; muscle weakness; tremors; and any pathological condition, where cannabinoid uses can be useful

Dosage regimen of the formulation depends on the disease, symptom and/or condition, the size of the human patient, and/or the form (liquid, gel) of the formulation. In some examples, Cannabidiol can be given 50 mg to 700 mg two to three times a day rectally as long as the treatment is beneficial. In some other examples Cannabidiol can be given 200 mg to 500 mg two times a day rectally for up to 3 months. In some examples, the concentration of Cannabidiol is about 50 mg/mL to about 200 mg/mL.

### K. METHOD OF ADMINISTRATION

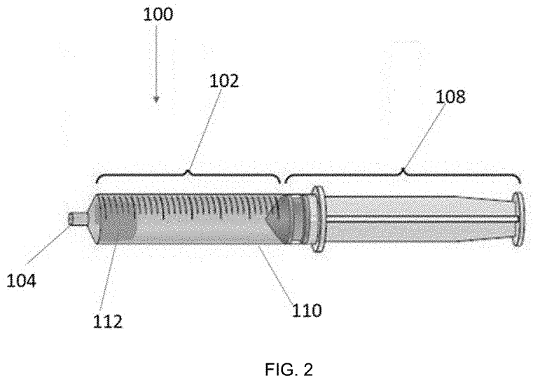

Formulations according to embodiments herein can be administered, i.e., delivered, to a non-oral cavity of a patient by using a suitable applicator. A preferred applicator, as shown in FIG. 2, is a syringe (100) that has a cylindrical syringe barrel (102) connected to an elongated, hollow delivery tip (106) that is designed for insertion into the non-oral cavity of the patient to be treated. In one embodiment, the syringe (100) has a hollow, open tip (104). In one embodiment, the barrel (102) is about 3 cm to about 8 cm long. The barrel 102 and/or the open tip (104) may be flexible. Preferably, the tip (104) is blunt enough to eliminate any risk of injury to patient tissue, when the tip (104) is inserted into non-oral cavity, yet small enough to for easy administration. In one embodiment, the applicator, e.g., syringe (100), is further mounted with a pressuring means, for example, a plunger (108) that can slide into a wall (110) of the applicator (100) to force out the formulation (112) through the hollow tip (104) into the non-oral cavity.

Preferably, the applicator (100) is made of a material that does not absorb or chemically react with the any components of the formulation. For example, the applicator (100) can be made of plastic, such as rigid polyethylene or polypropylene. Other materials, such as metal or glass can also be used. In a further embodiment, part of the applicator (100) can be made of plastic, and another part of metal or glass material. For example, a prefilled glass syringe or cartridge with gel composition can be placed into an applicator made of a plastic barrel, which prevents any breakage to glass syringe.

The applicator (100) can be disposable or reusable. If reusable, the applicator (100) is made of a material that can be sterilized by known acceptable medical sterilization methods.

In a preferred embodiment, a plastic applicator (100) is pre-packed as a medicated syringe-like assembly as a disposable applicator unit with a dose of the formulation according to an embodiment disclosed herein. The applicator (100) contains a suitable premeasured volume of a formulation according to an embodiment herein to be administered into a non-oral cavity of a human patient. While a single dose can be given using the applicator, the dose can be adjusted based on body weight, or whether the patient is an adult or a child, or based on indication. In one example, the entirety of the formulation (112) packed into the applicator (100) can be administered to a patient. In another example, only a part of the formulation (112) packed into the applicator (100) is administered to a patient and the remainder is discarded. In another embodiment, a portion of the formulation (112) is administered to a patient and the remainder is saved for administration to the same patient at a later time or date. The ability to administer all or a part of the formulation (112) to a patient allows the administration to be “adjustable”. Administration of the formulation (112) to a patient exhibiting symptoms will alleviate some or all of the symptoms for a particular amount of time.

In one embodiment, an applicator (100) is filled with volume of single unit or multi dose of the formulation (112) of about a 0.5 ml to about a 6 ml, which contains the therapeutically effective amount of the formulation. In one preferred embodiment, the applicator (100) is filled with a premeasured amount of the formulation, wherein applicator can be reused while the cartridge disposed after use.

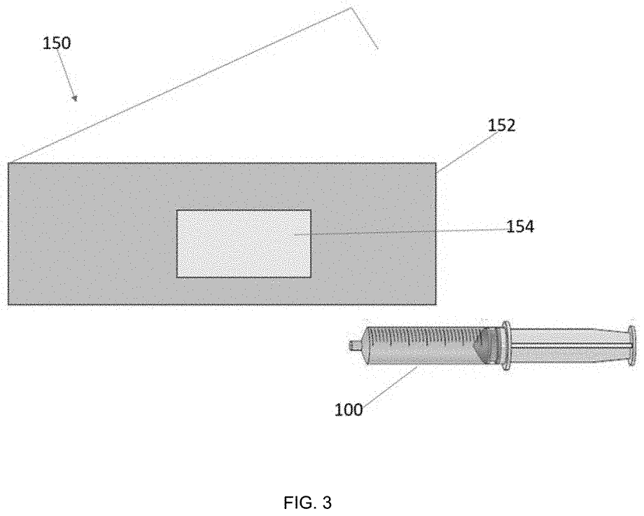

A preferred embodiment of the invention is a kit (150) as shown in FIG. 3. The kit (150) includes an applicator, e.g., a syringe, (100) pre-filled with a dose of premeasured amount of a formulation (112) according to an embodiment disclosed herein. The kit (150) is preferably contained in suitable packaging (152), such as a box, envelope or plastic blister pack, along with suitable “instructions of use” (154), such as a printed label, tag, or package.

### L. PREPARATION OF THE FORMULATION—EXAMPLES

The formulation according to embodiments of the invention can be formulated by mixing the ingredients according to generally accepted procedures for formulating pharmaceutical formulations. Generally, the ingredients can be mixed in a blender, or other standard mixing device to produce a liquid or gel formulation. Adequate mixing and procedures for elimination of lumps is used to ensure that a homogenous viscous gel is formed. A final concentration is achieved by adding water or organic solvent to the mixture. Similarly, adequate mixing is used to ensure that all components have fully dissolved and dispersed to form a liquid formulation.

If applicable, the quantities of the buffering agents (pH adjusting agents) are adjusted to obtain the desired pH in the gel formulation. Alternatively, if the required amounts of the all components have been calculated by prior experiments, these amounts can be added and mixed together in a mixing vessel, with no post adjustment of pH.

In general, and unless otherwise noted, the preparation of the sample formulations was conducted at room temperature.

### Example 1.—Aqueous Gel Formulation

An aqueous gel formulation was prepared using the components listed in Table 1.

TABLE 1

| Components | Category | Quantity | % w/w |
|---|---|---|---|
| Cannabidiol | Active | 15 g | 10.00 |
| Hydroxypropyl methyl cellulose (E50 LV) | Thickener | 6.34 g | 4.23 |
| Propylene glycol | Co-solvent | 47.61 g | 31.74 |
| Dehydrated alcohol | Co-solvent | 52.55 g | 35.03 |
| Benzyl alcohol | Preservative | 2.24 g | 1.49 |
| Benzoic acid | pH adjusting | 0.2 g | 0.13 |
| | agent | | |
| Water | Solvent | 26.06 g | 17.37 |
| Total | — | 150 g | 100.00 |

Preparation:

Required quantity of propylene glycol, polyethylene glycol and dehydrated alcohol were collected in a clean stainless-steel vessel and mixed at 200 rpm at room temperature for 5 minutes. Required quantity of benzoic acid was added to the stainless-steel vessel and mixed at 200 rpm at room temperature for 10 minutes to dissolve. 15 gm of Cannabidiol was added to the stainless-steel vessel and mixed at 200 rpm at room temperature for 15 minutes to dissolve. Required quantity of hydroxypropyl methyl cellulose (E 50 LV) was added to the stainless-steel vessel and mixed at 200 rpm at room temperature for 10 minutes to disperse. Required quantity of water was added to the stainless-steel vessel and mixed at 200 rpm at room temperature for about 15 min to form a clear, colorless to slight yellow viscous gel.

### Example 2

An aqueous gel formulation was prepared using the components listed in Table 2.

TABLE 2

| Components | Category | Quantity | % w/w |
|---|---|---|---|
| Cannabidiol | Active | 15 g | 10.00 |
| Hydroxypropyl methyl cellulose (E50 LV) | Thickener | 6.34 g | 4.22 |
| Polyethylene glycol 400 | Co-solvent | 15 g | 10.00 |
| Propylene glycol | Co-solvent | 32.61 g | 21.74 |
| Dehydrated alcohol | Co-solvent | 52.55 g | 35.03 |
| Benzyl alcohol | Preservative | 2.24 g | 1.49 |
| Benzoic acid | pH adjusting | 0.2 g | 0.14 |
| | agent | | |
| Water | Solvent | 26.09 g | 17.39 |
| Total | — | 150 g | 100.00 |

Preparation:

Required quantity of propylene glycol, polyethylene glycol and dehydrated alcohol (purged with nitrogen) were collected in a clean stainless-steel vessel and mixed at 200 rpm for 5 minutes at room temperature. Required quantity of benzoic acid was added to the stainless-steel vessel and mixed at 200 rpm for 10 minutes to dissolve. 15 gm of Cannabidiol was added to the stainless-steel vessel and mixed at 200 rpm for 15 minutes to dissolve. Required quantity of hydroxypropyl methyl cellulose (E 50 LV) was added to the stainless-steel vessel and mixed at 200 rpm for 10 minutes to disperse. Required quantity of water (purged with nitrogen) was added to the stainless-steel vessel and mixed at 200 rpm for about 15 min to form clear, colorless to slight yellow viscous gel.

### Example 3

An aqueous gel formulation was prepared using the components listed in Table 3.

TABLE 3

| Components | Category | Quantity | % w/w |
|---|---|---|---|
| Cannabidiol | Active | 20.000 gm | 20.37 |
| Hydroxypropyl methyl cellulose (E50 | Thickener | 3.900 gm | 3.97 |
| LV) | | | |
| Polyethylene glycol 400 | Co-solvent | 14.600 gm | 14.87 |
| Propylene glycol | Co-solvent | 13.200 gm | 13.44 |
| Dehydrated alcohol | Co-solvent | 30.100 gm | 30.65 |
| Benzyl alcohol | Preservative | 1.300 gm | 1.32 |
| Benzoic acid | pH adjusting | 0.100 gm | 0.10 |
| | agent | | |
| Water | Solvent | 15.000 gm | 15.27 |
| Total | — | 98.200 gm | 100.00 |

Process:

Required quantity of propylene glycol, polyethylene glycol and dehydrated alcohol were collected in a glass vessel and mixed at 200 rpm for 5 minutes at room temperature. Required quantity of benzoic acid was added to the glass vessel and mixed at 200 rpm for 10 minutes to dissolve. 20 gm of Cannabidiol was added to the glass vessel and mixed at 200 rpm for 15 minutes to dissolve. Required quantity of Hydroxypropyl methyl cellulose (E 50 LV) was added to the glass vessel and mixed at 600 rpm for 10 minutes to disperse. Required quantity of water was added to the glass vessel and mixed at 600 rpm for about 30 min to form clear, colorless to slight yellow viscous gel.

### Example 4

An aqueous gel formulation was prepared using the components in Table 4, including a penetration enhancer.

TABLE 4

| Components | Category | Quantity | % w/w |
|---|---|---|---|
| Cannabidiol | Active | 20.000 g | 20.42 |
| N-Methyl-2-Pyrrolidone (NMP) | Penetration enhancer | 9.157 g | 9.35 |
| Hydroxypropyl methyl | Thickener | 3.897 g | 3.98 |
| cellulose (E50 LV) | | | |
| Polyethylene glycol 400 | Co-solvent | 14.613 g | 14.92 |
| Propylene glycol | Co-solvent | 4.059 g | 4.14 |
| Dehydrated alcohol | Co-solvent | 30.200 g | 30.84 |
| Benzoic acid | pH adjusting agent | 0.117 g | 0.12 |
| Benzyl alcohol | Preservative | 1.286 g | 1.31 |
| Water | Solvent | 14.613 g | 14.92 |
| Total | — | 97.942 g | 100.00 |

Preparation:

Required quantity of propylene glycol, polyethylene glycol, dehydrated alcohol and N-Methyl-2-Pyrrolidone (NMP) were collected in a glass vessel and mixed at 200 RPM for 5 minutes. Required quantity of benzoic acid was added to the glass vessel and mixed at 200 RPM for 10 minutes to dissolve. Required quantity of Cannabidiol was added to the glass vessel and mixed at 200 RPM for 15 minutes to dissolve. Required quantity of Hydroxypropyl methyl cellulose (E 50 LV) was added to the glass vessel and mixed at 600 RPM for 10 minutes to disperse. Required quantity of water was added to the glass vessel and mixed at 600 RPM for about 30 min to form a clear, colorless to slight yellow color viscous gel.

### Example 5

An aqueous gel formulation was prepared using the components in Table 5, with a penetration enhancer.

TABLE 5

| Components | Category | Qty/mL | % w/w |
|---|---|---|---|
| Cannabidiol | Active | 20.000 g | 20.42 |
| Salcaprozic acid | Penetration enhancer | 2.597 g | 2.65 |
| Hydroxypropyl methyl cellulose | Thickener | 3.895 g | 3.98 |
| (E50 LV) | | | |
| Polyethylene glycol 400 | Co-solvent | 14.606 g | 14.91 |
| Propylene glycol | Co-solvent | 10.776 g | 11.00 |
| Dehydrated alcohol | Co-solvent | 30.185 g | 30.82 |
| Benzyl alcohol | Preservative | 1.285 g | 1.31 |
| Water | Solvent | 14.606 g | 14.91 |
| Total | — | 97.948 g | 100.01 |

Preparation:

Required quantity of propylene glycol, polyethylene glycol and dehydrated alcohol were collected in a glass vessel and mixed at 200 RPM for 5 minutes. Required quantity of salcaprozic acid and benzoic acid was added to the glass vessel and mixed at 400 RPM for 20 minutes to dissolve. Required quantity of Cannabidiol was added to the glass vessel and mixed at 300 RPM for 15 minutes to dissolve. Required quantity of Hydroxypropyl methyl cellulose (E 50 LV) was added to the glass vessel and mixed at 600 RPM for 10 minutes to disperse. Required quantity of water was added to the glass vessel and for mixed at 600 RPM for about 30 min to form a clear, colorless to slight yellow color viscous gel.

### Example 6

A microemulsion formulation was prepared using the components in Table 6.

TABLE 6

| Components | Category | Quantity | % w/w |
|---|---|---|---|
| Cannabidiol | Active | 1.6 gm | 8% |
| Polyoxyl-40 hydrogenated castor | Surfactant | 850 mg | 4.25% |
| oil | | | |
| Glycerol/glyceryl monolinoleate | Oil phase | 1058 mg | 5.29% |
| Propylene glycol | Co-solvent | 160 mg | 0.8% |
| Dehydrated alcohol | Co-solvent | 320 mg | 1.6% |
| Water | Solvent | 16 gm | 80% |
| Total | — | 20 gm | 100% |

Preparation:

Required quantity of Polyoxyl-40 hydrogenated castor oil was melted at about 40° C. and dispensed into a glass container. Glycerol/glyceryl monolinoleate, propylene glycol and alcohol were added to the glass container, mixed at 200 RPM for 10 minutes and the mixture was allowed to cool to room temperature. 1.6 gm of Cannabidiol was added to the glass container and mixed at 200 RPM for 10 minutes to dissolve. A clear slight yellow solution was obtained. The prepared solution was transferred in separate glass container containing the required quantity of water with continuous mixing at 600 RPM for 15 minutes. A milky dispersion was formed.

### Example 7

A microemulsion formulation was prepared using the components in Table 7, with an anti-oxidant.

TABLE 7

| Components | Category | Qty/gm | % w/w |
|---|---|---|---|
| Cannabidiol | Active | 1.6 gm | 8% |
| Polyoxyl-40 hydrogenated castor oil | Surfactant | 850 mg | 4.25% |
| Glycerol/glyceryl monolinoleate | Oil phase | 1058 mg | 5.29% |
| Propylene glycol | Co-solvent | 160 mg | 0.8% |
| Dehydrated alcohol | Co-solvent | 320 mg | 1.6% |
| DL-α-Tocopherol | Anti-oxidant | 12 mg | 0.06% |
| Water | Solvent | 16 gm | 80% |
| Total | — | 20 gm | 100% |

Preparation:

Required quantity of Polyoxyl-40 hydrogenated castor oil was melted at about 40° C. and dispensed in glass container. Glycerol/glyceryl monolinoleate, PG and alcohol were added to the glass container, mixed at 200 RPM for 10 minutes and allowed to cool to room temperature. Required quantity of tocopherol was added to the glass container and mixed at 200 RPM for 10 minutes. 1.6 gm of Cannabidiol was added to the glass container and mixed at 200RPM for 10 minutes to dissolve. A clear slight yellow solution was obtained. The prepared solution was transferred into a separate glass container containing water with continuous mixing at 600 RPM for 15 minutes. A milky dispersion was formed. The milky dispersion was further homogenized at 4000 rpm for 1 min.

### Example 8

A self-emulsifying solution formulation was prepared with the components shown in Table 8.

TABLE 8

| Components | Category | Quantity | % w/w |
|---|---|---|---|
| Cannabidiol | Active | 12.0 gm | 40.0% |
| Polyoxyl-40 hydrogenated | Surfactant | 6.375 gm | 21.25% |
| castor oil | | | |
| Glycerol/glyceryl monolinoleate | Oil phase | 7.935 gm | 26.45% |
| Propylene Glycol | Co-solvent | 1.2 gm | 4% |
| Dehydrated Alcohol | Co-solvent | 2.4 gm | 8% |
| DL-α-Tocopherol | Anti-oxidant | 90 mg | 0.3% |
| Total | — | 30.0 gm | 100% |

Preparation:

Required quantity of Polyoxyl-40 hydrogenated castor oil was melted at about 40° C. and dispensed into a glass container. Glycerol/glyceryl monolinoleate, propylene glycol and alcohol were added to the glass container, mixed at 200 RPM for 10 minutes and allowed to cool to room temperature. Required quantity of tocopherol was added to the glass container and mixed at 300 RPM for 15 minutes. 12 gm of Cannabidiol was added to the glass container and mixed at 400 RPM for 15 minutes to dissolve. A clear slight yellow solution was obtained.

### Example 9

A microemulsion formulation was prepared with the components listed in Table 9.

TABLE 9

| Components | Category | Qty/gm | % w/w |
|---|---|---|---|
| Cannabidiol | Active | 3.2 gm | 16.00 |
| Polyoxyl-40 hydrogenated castor oil | Surfactant | 1.7 gm | 8.50 |
| Glycerol/glyceryl monolinoleate | Oil phase | 2.116 gm | 10.58 |
| Propylene glycol | Co-solvent | 320 mg | 1.60 |
| Dehydrated alcohol | Co-solvent | 640 mg | 3.20 |
| DL-α-Tocopherol | Anti-oxidant | 24 mg | 0.12 |
| Water | Solvent | 12 gm | 60.00 |
| Total | — | 20 gm | 100.00 |

Preparation:

Required quantity of Polyoxyl-40 hydrogenated castor oil was melted at about 40° C. and dispensed into a glass container. Glycerol/glyceryl monolinoleate, PG and alcohol were added to the glass container, mixed at 300 RPM for 10 minutes and allowed to cool to room temperature. Required quantity of tocopherol was added to the glass container and mixed at 300 RPM for 10 minutes. 3.2 gm of Cannabidiol was added to the glass container and mixed at 400 RPM for 15 minutes to dissolve. A clear slight yellow solution was obtained. The prepared solution was transferred into a separate glass container containing water with continuous mixing at 600 RPM for 10 minutes. A milky dispersion was formed. The milky dispersion was further homogenized at 4000 rpm for 1 min.

### Example 10

A viscous solution formulation prepared from the components in Table 10.

TABLE 10

| Components | Category | Quantity | % w/w |
|---|---|---|---|
| Cannabidiol | Active | 6.000 gm | 30.00 |
| Polyoxyl-40 hydrogenated castor oil | Surfactant | 13.800 gm | 69.00 |
| Benzoic acid | pH adjusting agent | 100 mg | 0.50 |
| DL-α-Tocopherol | Anti-oxidant | 100 mg | 0.50 |
| Total | — | 20.000 gm | 100.00 |

Preparation:

Required quantity of Polyoxyl-40 hydrogenated castor oil was melted at about 40° C. in a glass vessel. Required quantity of benzoic acid and tocopherol were added to the glass vessel and dissolved. 6 gm of Cannabidiol was added to the glass vessel and mixed at 300 RPM for 10 minutes to dissolve. A clear solution was formed. The clear solution became thick and viscous with very slight yellow color.

### Example 11

An emulgel formulation was prepared with the components in Table 11.

TABLE 11

| Components | Category | Quantity | % w/w |
|---|---|---|---|
| Cannabidiol | Active | 3.2 gm | 16.00 |
| Polyoxyl-40 hydrogenated | Surfactant | 1.7 gm | 8.50 |
| castor oil | | | |
| Glycerol/glyceryl monolinoleate | Oil phase | 2.116 gm | 10.58 |
| Propylene Glycol | Co-solvent | 0.320 gm | 1.60 |
| Dehydrated Alcohol | Co-solvent | 0.640 gm | 3.20 |
| DL-α-Tocopherol | Anti-oxidant | 24 mg | 0.12 |
| Hydroxypropyl methyl | Thickener | 0.600 gm | 3.00 |
| cellulose (E 50 LV) | | | |
| Water | Solvent | 11.400 gm | 57.00 |
| Total | — | 20.00 gm | 100.00 |

Preparation:

Step 1: Required quantity of Glycerol/glyceryl monolinoleate and Polyoxyl-40 hydrogenated castor oil are collected in glass container. Required quantity of alcohol, tocopherol and propylene glycol were added to the glass container and mixed at 300 RPM for 10 minutes. 3.2 gm of Cannabidiol was added to the glass container and dissolved.

Step 2: In separate glass container, required quantity of water was heated to about 60° C. and Hydroxypropyl methyl cellulose was dispersed. The dispersion was cooled to about 40° C. and the step 1 solution was gradually added with mixing at 600 RPM for 10 minutes. The resulting dispersion was milky white and thickened at room temperature.

### Stability Results

| Example# | Condition | Description | Assay |
|---|---|---|---|
| 1 | Initial | — | 102.4% |
| | 1M/40° C./75% RH | Clear colorless Gel | 108.6% |
| | 2M/40° C./75% RH | Clear colorless viscous gel | 103.8% |
| | 3M/40° C./75% RH | Pale Yellow color viscous gel | 100.8% |
| | 3M/25° C./60% RH | Pale Yellow color viscous gel | 100.7% |
| 2 | Initial | — | 105.2% |
| | 1M/40° C./75% RH | Clear colorless Gel | 106.7% |
| | 2M/40° C./75% RH | Clear colorless viscous gel | 106.3% |
| | 3M/40° C./75% RH | Pale Yellow color viscous gel | 102.9% |
| | 3M/25° C./60% RH | Colorless to pale Yellow | 103.9% |
| | | color viscous gel | |
| 3 | Initial | Clear yellowish viscous gel | 102.9% |
| 4 | Initial | Pale yellowish viscous gel | 102.9% |
| 5 | Initial | Pale yellowish viscous gel | 102.3% |

The formulation was tested for condition and assay at initial (before loading on stability) and after 1, 2 and 3 months at 25° C./60% RH and 40° C./75% RH stability conditions. “Condition” is visual description while “assay” is performed using HPLC method. The Assay is tested against the labeled concentration and it reflects the potency of the drug product. It was observed that even after 3 months at 40° C./75% RH which is accelerated condition, potency of the product was maintained.

As will be apparent to those skilled in the art, various modifications, adaptations and variations of the foregoing specific disclosure can be made without departing from the scope of the invention claimed herein. The various features and elements of the invention described herein may be combined in a manner different than the specific examples described or claimed herein without departing from the scope of the invention. In other words, any element or feature may be combined with any other element or feature in different embodiments, unless there is an obvious or inherent incompatibility between the two, or it is specifically excluded.

References in the specification to “one embodiment,” “an embodiment,” etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such aspect, feature, structure, or characteristic with other embodiments, whether or not explicitly described.

The singular forms “a,” “an,” and “the” include plural reference unless the context clearly dictates otherwise. Thus, for example, a reference to “a plant” includes a plurality of such plants. It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition or step being referred to is an optional (not required) feature of the invention.

The term “and/or” means any one of the items, any combination of the items, or all of the items with which this term is associated. The phrase “one or more” is readily understood by one of skill in the art, particularly when read in context of its usage.

Each numerical or measured value in this specification is modified by the term “about”. The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values and ranges proximate to the recited range that are equivalent in terms of the functionality of the composition, or the embodiment.

As will be understood by the skilled artisan, all numbers, including those expressing quantities of reagents or ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

What is claimed:

1. A pharmaceutically acceptable gel formulation of cannabinoid for non-oral delivery comprising:
(a) at least one cannabinoid at 0.05% to 50% by weight of the total formulation;
(b) optionally a pH adjusting agent in a concentration effective to maintain the pH of the formulation at about 5-8;
(c) a thickener in a concentration effective to impart a viscosity to the formulation in a range of 1,000 to 8,000 cP so that it is suitable for administration by applicator or syringe like injector to a human patient; and
(d) a solvent at 10% to 95% by weight of the total formulation.

2. The formulation of claim 1, wherein the at least one cannabinoid is selected from the group consisting of CBD, CBDA, CBDV, THC, THCA, THCV, CBG, CBN, and combinations thereof.

3. The formulation of claim 2, wherein the at least one cannabinoid is CBD and wherein CBD comprises 0.1% to 20% by weight of the total formulation.

4. The formulation of claim 2, wherein the at least one cannabinoid is THC and wherein THC comprises 0.05% to 2% by weight of the total formulation.

5. The formulation of claim 2, wherein the at least one cannabinoid comprises a mixture of CBD and THC, wherein CBD comprises 0.1% to 20% by weight of the total formulation, and THC comprises 0.05% to 2% by weight of the total formulation.

6. The formulation of claim 1 wherein the concentration of the thickener is 0.05 to 10% by weight of the total formulation.

7. The formulation of claim 1 wherein the thickener is selected from the group consisting of a cellulose ether; a polyacrylic acid polymer (carbomer); natural gum, and combinations thereof.

8. The formulation of claim 7, wherein the cellulose ether is selected from the group consisting of hydroxypropyl methylcellulose, methylcellulose, hydroxypropyl cellulose, carboxyl methylcellulose, and combinations thereof.

9. The formulation of claim 8 wherein the thickener is hydroxypropyl methylcellulose.

10. The formulation of claim 1 wherein the solvent is water or a mixture of water and at least one organic solvent.

11. The formulation of claim 1, further comprising a pharmaceutically acceptable excipient selected from group consisting of preservatives, buffers, inclusion complexing agents, surfactants and combinations thereof.

12. The formulation of claim 1, wherein the at least one cannabinoid is suspended in the gel composition.

13. The formulation of claim 12, wherein a size of the suspended cannabinoid is between 10 Nanometer to 50 Micrometer.

14. The formulation of claim 1, wherein the formulation is suitable for administration via a rectal route of administration.

15. The formulation of claim 1 packaged in a suitable rectal applicator.

16. The formulation of claim 1, wherein the formulation is suitable for administration in a non-oral cavity of a patient.

17. The formulation of claim 1, wherein the formulation is suitable for transdermal application.

18. A pharmaceutically acceptable liquid or viscous liquid formulation of cannabinoid for non-oral delivery comprising:
a. at least one cannabinoid at 0.05% to 50% by weight of the total formulation;
b. optionally an antioxidant;
c. optionally a pH adjusting agent; and
d. optionally an alcohol-based co-solvent
wherein the formulation is suitable for rectal or vaginal administration.

19. A liquid or viscous liquid formulation of claim 18, further comprising:
a pharmaceutical excipient; or
a hydrogenated oil at 10% to 95% by weight of the total formulation.

20. A liquid or viscous liquid formulation of claim 18, wherein the at least one cannabinoid is selected from the group consisting of CBD, CBDA, CBDV, THC, THCA, THCV, CBG, CBN and combinations thereof.

21. A liquid or viscous liquid formulation of claim 20, wherein the at least one cannabinoid is:
CBD;
THC; or
a combination of CBD and THC.

22. A kit for administering a formulation of cannabinoid for non-oral delivery, the kit comprising:
a syringe assembly pre-filled with a dose of a formulation according to claim 1; and
instructions of use,
wherein the syringe assembly and instructions are placed in packaging.

23. A kit for administering a formulation of cannabinoid for non-oral delivery, the kit comprising:
a syringe assembly pre-filled with a dose of a formulation according to claim 18; and
instructions of use,
wherein the syringe assembly and instructions are placed in packaging.

24. A method for administering a formulation of cannabinoid to alleviate symptoms exhibited by a patient, the method comprising:
providing a formulation of cannabinoid, the formulation comprising: at least one cannabinoid at 0.05% to 50% by weight of the total formulation; and optionally a pH adjusting agent in a concentration effective to a maintain the pH of the formulation at about 5-8; and
delivering the formulation to a non-oral cavity of the patient,
wherein after said delivery of the gel formulation, the symptoms of the patient are alleviated.
